(19) United States
(12) Patent Application Publication
(10) Pub. No.: US 2026/0159472 A1
(43) Pub. Date: Jun. 11, 2026

(54) PROCESS FOR PREPARING SECONDARY AND/OR TERTIARY AMINES IN THE PRESENCE OF A MANGANESE-DOPED COPPER CATALYST

(71) Applicant: Arkema France, Colombes (FR)

(72) Inventor: Christophe RUPPIN, La Chambre (FR)

(73) Assignee: Arkema France, Colombes (FR)

(21) Appl. No.: 18/722,138

(22) PCT Filed: Dec. 19, 2022
(86) PCT No.: PCT/FR2022/052424

§ 371 Date: Jun. 20, 2024

(30) Foreign Application Priority Data
Dec. 21, 2021 (FR) FR2114157

(51) Int. Cl. C07C 209/26; C07C 209/16

(52) U.S. Cl. CPC C07C 209/26; C07C 209/16

(57) ABSTRACT

The present invention relates to a process for preparing secondary and/or tertiary amines, comprising an aminating step, said aminating step being carried out by gas-phase reaction of a primary or secondary alcohol and/or of a ketone with ammonia or a primary or secondary amine, in the presence of a catalyst,
said catalyst comprising copper and being doped with manganese, the amount of manganese being between 1% and 10% by weight, relative to the total weight of the catalyst.

The present invention relates to a process for synthesizing secondary and/or tertiary amines by gas-phase reaction of a primary or secondary alcohol and/or of a ketone with ammonia or a primary or secondary amine, in the presence of a catalyst comprising copper as catalytically active metal, doped with manganese.

The present invention also relates to the use of such a catalyst for the synthesis of secondary and/or tertiary amines.

Amines, and especially alkylamines, are organic compounds with very diverse industrial applications. These compounds are used in particular as neutralizing agents, corrosion inhibitors, polymerization and/or crosslinking catalysts, and especially as synthesis intermediates in pharmacy, agrochemistry, electronics and detergency.

Possible examples of such compounds include:
diisopropylamine (DIPA), a secondary amine, which is the principal synthetic precursor to N-ethyldiisopropylamine (Hünig's base), which is used as an acid scavenger when synthesizing pharmaceutical or agrochemical active principles. DIPA is also an access point for diisopropylaminosilane (DIPAS) and other volatile aminosilane derivatives, which are precursors of choice for the controlled deposition of silicon oxide or silicon nitride films in the fabrication of semiconductor devices;
N-ethylmethylamine (EMA), which is involved in the manufacture of active pharmaceutical molecules intended for treating degenerative diseases of the nervous system and in the synthesis of metal salts, as for example tetrakis(ethylmethylamino) hafnium or -zirconium compounds, which are volatile precursors of choice for the production of deposited metallic films by CVD (Chemical Vapour Deposition) or ALD (Atomic Layer Deposition) in the fabrication of semiconductors; and
N,N-dimethylethylamine (DMEA) and N,N-dimethylisopropylamine (DMIPA), which are tertiary amines used as polymerization catalysts for polyurethane resins for the manufacture of foundry moulds by the “cold box” process.

The preparation of secondary and/or tertiary amines by amination of alcohols and/or ketones with ammonia or primary or secondary amines in the presence of hydrogen and a hydrogenation/dehydrogenation catalyst is widely known (Amines, Aliphatic, sections 3.1 and 3.2, Ullmann's Encyclopedia of Industrial Chemistry, 2015, Wiley Online Library). The preparation proceeds in liquid phase or in gaseous phase according to the nature of the starting materials and/or the type of catalyst.

One possible synthesis example is that of diisopropylamine (DIPA), which is conventionally obtained as a by-product in processes for manufacturing monoisopropylamine (MIPA) by catalytic amination of acetone and/or isopropanol with ammonia.

From acetone, the synthesis is performed according to the reaction scheme below:

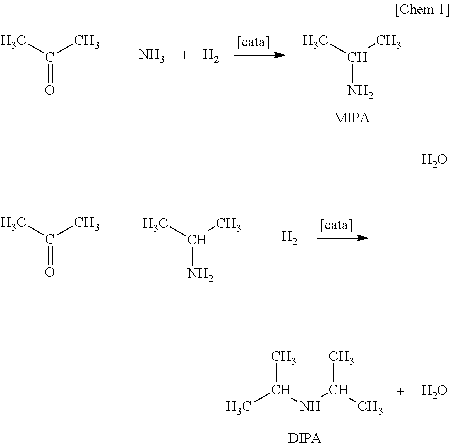

From isopropanol, the reactions are similar, after prior in situ dehydrogenation of the isopropanol to acetone:

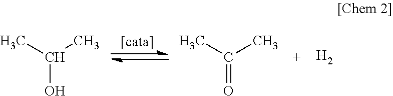

This synthesis is usually performed continuously via gas-phase or liquid-phase processes which lead to the production predominantly or even almost exclusively of MIPA, for which the principal application worldwide remains glyphosate salt.

For selective acquisition of DIPA, the latter may be produced directly starting from MIPA and acetone as starting reactants, or else MIPA is subjected generally to continuous dismutation at high temperature through a fixed bed of a catalyst or a zeolite, according to the following scheme:

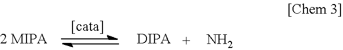

It is therefore apparent that the most selective syntheses of DIPA, and of secondary amines in general, involve implementing the amination of acetone (aldehyde or ketone more generally) or of isopropanol (alcohol more generally) with primary amines (MIPA in the case of DIPA) or else carrying out the dismutation of the latter compounds. These two techniques thus require the prior manufacture of primary amines as principal products, followed by conversion into secondary amines.

There is therefore a need for a process for synthesizing secondary and/or tertiary amines as principal products (and no longer as by-products), especially starting from ketone and/or alcohol and ammonia for the synthesis of secondary amines.

There is a need for a process for synthesizing secondary and/or tertiary amines that is easy to implement and is industrially viable.

Moreover, in instances where ketones (especially acetone) are less expensive than the corresponding alcohols (especially isopropanol), ketones are the preferred starting materials. The reductive amination of ketones with ammonia or a primary or secondary amine, though, is a much more exothermic process than the reductive amination of alcohols. When the amination reaction of a ketone is performed continuously through a fixed catalytic bed, this high level of heat produced generates a substantial increase in temperature within the grains of the catalyst, giving rise to secondary reactions and therefore a loss of selectivity, or else imposes a need to operate at a lower volume flow rate of ketone per unit volume of catalyst (LHSV), with a consequent loss in productivity relative to the same process conducted starting from alcohol. This higher temperature may also have a negative impact on the ageing and hence the lifetime of the catalyst.

Accordingly, a high temperature and/or a substantial excess of nitrogenous reactant may result in the formation of undesirable aminic impurities. The impurities formed notably include those originating from secondary reactions of transamination or dismutation which result in the formation of unwanted amines.

For example and not exhaustively, in the case of DIPA, the breakdown of acetone to acetaldehyde leads in particular to the formation of monoethylamine (MEA) and N-ethylisopropylamine (EIPA) according to the following scheme:

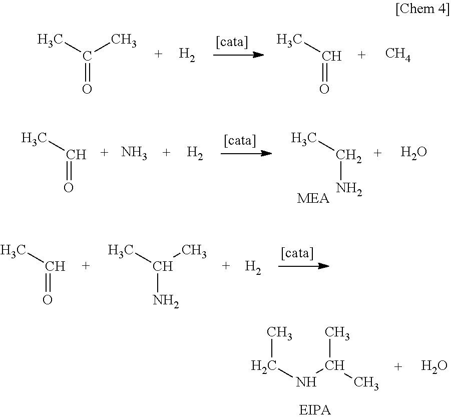

and/or

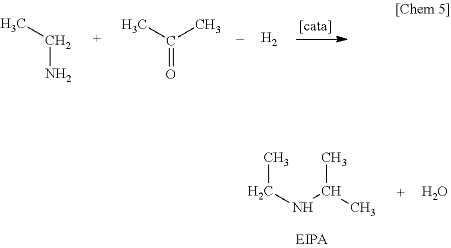

In addition, the parasitic self-condensation of acetone leads to the formation of methyl isobutyl ketone (MIBK), which by reductive amination with ammonia and with monoisopropylamine leads respectively to the secondary formation of 1,3-dimethylbutylamine (1,3-DMBA) and N-(1,3-dimethylbutyl) isopropylamine (DMBIPA).

Likewise, where dimethylamine (DMA) is used for manufacturing tertiary amines of dimethylalkylamine type such as DMEA, DMIPA or DMPA, the DMA may be partially dismutated to trimethylamine (TMA) and monomethylamine (MMA), according to the reaction below:

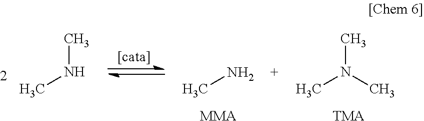

The MMA may also react with the alcohol or the ketone to form secondary amines, which are difficult to separate from the desired amines.

Where MMA is used for manufacturing secondary amines of alkylmethylamine type such as N-ethylmethylamine (EMA) or N-isopropylmethylamine, the MMA may be partially dismutated to dimethylamine (DMA) and ammonia, according to the reaction:

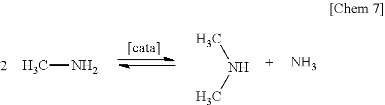

In the manufacture of EMA starting from ethanol, the by-product DMA may then react with the ethanol to form DMEA, whose boiling point is very close to that of EMA (36.5 as against 32.6° C.), thus making it very complex to purify the EMA to meet the required specifications, especially for electronics applications. The ammonia may also react with the ethanol to lead to mono-, di- and/or triethylamine by-products.

It is therefore apparent that the current amination reactions give rise to the formation of numerous aminic impurities. These impurities make it particularly difficult to obtain secondary and/or tertiary amines of satisfactory, let alone high, purity.

There is therefore also a need for a process for synthesizing secondary and/or tertiary amines that is selective for desired secondary or tertiary amines, and particularly that limits or prevents the formation of aminic impurities.

One objective of the present invention is to provide a process for synthesizing secondary and/or tertiary amines that is simple and industrially viable.

Another objective of the present invention is to provide a gas-phase process for synthesizing secondary and/or tertiary amines that is easy to implement.

A further objective of the present invention is to provide a selective process for synthesizing secondary or tertiary amines, preferably secondary amines.

One objective of the present invention is to provide an amination catalyst enabling the acquisition of satisfactory or even high selectivity for secondary or tertiary amines.

One objective of the present invention is to provide an amination catalyst that limits or even prevents the amine transamination or dismutation reactions and hence the formation of aminic impurities.

The present invention meets all or part of the objectives above.

The present inventor has found a new process for preparing amines using a catalyst enabling the acquisition of satisfactory or even high or improved conversion and/or selectivity for secondary and/or tertiary amines. The new catalyst limits or even prevents the formation of aminic impurities, especially those formed by amine transamination or dismutation. The reaction thus catalysed is improved and easy to implement. The secondary and/or tertiary amines formed according to the invention can be purified more easily.

The present inventor surprisingly has also found a process for synthesizing secondary amines that is highly selective, especially when the catalyst according to the invention is used and when the co-produced primary and/or tertiary amines are recycled to the aminating step. A process like this is able in particular to attain a selectivity for secondary amines of more than 90%.

In particular, the process according to the invention enables the selective synthesis of secondary amines directly starting from alcohol and/or ketone and ammonia.

Accordingly, the present invention relates to a process for preparing secondary and/or tertiary amines, comprising an aminating step, said aminating step being carried out by gas-phase reaction of a primary or secondary alcohol and/or of a ketone with ammonia or a primary or secondary amine, in the presence of a catalyst and hydrogen,
said catalyst comprising copper doped (or promoted) with manganese, and the amount of manganese being between 1% and 10% by weight, relative to the total weight of the catalyst.

The present invention also relates to the use of a catalyst comprising copper doped with manganese, the manganese being present in an amount of between 1% and 10% by weight, relative to the total weight of the catalyst, for preparing secondary and/or tertiary amines.

### Definitions

According to the present invention, “catalyst” refers to the catalytic composition comprising the active metals and the dopants (in particular, copper and manganese, in whatever form, oxidized or otherwise) and also the support and any additives. The weight percentages stated below correspond to the catalyst before any pre-activation or activation.

It is understood that in the catalyst according to the invention, the copper is the active metal and the manganese is a dopant. A “dopant” (also known as a “promoter”) refers to a chemical substance or a composition of chemical substances which can modify and in particular improve the catalytic activity of a catalyst. For example, a “dopant” refers to a chemical substance or a composition of chemical substances for improving the conversion and/or selectivity of the catalysed reaction relative to the catalyst without dopant.

A “nitrogenous reactant” refers to ammonia and/or the primary or secondary amines used as reactants in the amination reaction as according to the invention.

The “selectivity” is S_{A} or the selectivity for amine (A) produced relative to the reactants converted, calculated according to the following equation:

$${S}_{A} = 100 \times ( {Z}_{reactant} / {Z}_{amine} ) \times ( number of moles of target amine formed / number of moles of reactant converted ) ,$$

where Zₐₘᵢₙₑ is the stoichiometric coefficient of the amine and Z_{reactant} is the stoichiometric coefficient of the reactant. The reactant used for the above calculation is preferably the limiting reactant.

In particular, the process according to the invention enables the acquisition of a selectivity for secondary amines of greater than or equal to 50%, for example of between 50% and 90%, preferably between 70% and 90%.

In particular, the process according to the invention enables the acquisition of a selectivity for tertiary amines of between 90% and 100%, preferably between 90% and 99%.

“Aminic impurity” refers notably to any unwanted primary, secondary or tertiary amine obtained following a parasitic reaction of transamination or dismutation or following a self-condensation of the ketone. The aim in particular is to limit or even prevent the formation of these impurities.

### Catalyst According to the Invention

The catalyst according to the invention comprises copper doped with manganese, the amount of manganese being between 1% and 10% by weight, relative to the total weight of the catalyst.

The amount of copper in the catalyst is preferably not more than 60% by weight, relative to the total weight of the catalyst. In particular, the amount of copper is between 15% and 60% by weight, relative to the total weight of the catalyst. The amount of copper is in particular between 20% and 60% by weight, preferably between 35% and 50% by weight, more preferably between 40% and 50% by weight, for example between 44% and 48% by weight, relative to the total weight of the catalyst. The copper may be present in the form of one or more copper oxides, preferably in the form of CuO.

The amount of manganese is preferably between 4% and 10% by weight, more preferably between 4% and 8% by weight, relative to the total weight of the catalyst. The manganese may be present in the form of one or more oxides, preferably in the form of manganese dioxide (MnO₂) or Mn₃O₄.

The catalyst may also comprise a support selected from the group consisting of alumina (Al₂O₃), silica (SiO₂), titanium dioxide, zirconia, and mixtures of two or more thereof, preferably alumina and/or silica.

In particular, the catalyst comprises:
between 20% and 60% by weight, preferably between 35% and 50%, for example between 40% and 50% by weight of copper, relative to the total weight of the catalyst;
between 1% and 10% by weight, preferably between 4% and 10%, for example between 4% and 8% by weight of manganese, relative to the total weight of the catalyst; and
alumina.

Said catalyst preferably comprises copper in the form of CuO and manganese in the form of MnO₂ and/or of Mn₃O₄. The copper and the manganese are present in particular in the form of one or more oxides before activation of the catalyst. Said catalyst preferably consists essentially, or even consists, of copper in oxidized form, of manganese in oxidized form, of a support such as alumina or silica and any additives.

More particularly, the catalyst comprises:
between 25% and 75% by weight, preferably between 40% and 65% by weight, of copper oxide (expressed as CuO), relative to the total weight of the catalyst; and
between 1% and 20% by weight, preferably between 5% and 15% by weight, of manganese oxides (expressed as MnO₂), relative to the total weight of the catalyst.

The catalyst preferably comprises no active metal other than the copper (i.e. whether in the elemental form or in the form of an organic or inorganic compound, for example a metallic oxide). The catalyst preferably comprises no dopant other than the manganese (i.e. whether in the elemental form or in the form of an organic or inorganic compound, for example a metallic oxide). In particular, said catalyst does not include chromium and/or nickel.

Preferably, the catalyst does not comprise rare earth metal. By rare earth metal, it is understood the scandium, the yttrium, and the lanthanides such as lanthanum, cerium, praseodymium, neodymium, and dysprosium. More particularly, the catalyst does not comprise cerium.

Preferably, the catalyst does not comprise element from the groups 8, 9 and 10 of the periodic table (formerly group VIII). In particular, the catalyst does not comprise platinum, palladium, ruthenium and rhodium. More particularly, the catalyst comprises neither rare earth metal nor element from the groups 8, 9 and 10 of the periodic table.

According to another embodiment, other metallic compounds may be included in the catalyst. Possible non-limiting examples of such compounds include molybdenum, tungsten, chromium, vanadium and magnesium. They may be in oxidic form, for example in the form of MoO₂, WO₂, Cr₂O₃, V₂O₅ and MgO.

The catalyst may also comprise other additives such as stabilizers and/or shaping auxiliaries such as graphite, which are customary in the field of catalysts. These compounds are included generally in an amount of between 1% and 15% by weight, relative to the total weight of the catalyst.

The catalyst is preferably used in the form of pellets having a diameter of between 3 and 6 mm and a length of between 3 and 6 mm.

One example that may be given is the catalyst HySat® 200 tab 4.8×4.8 from Clariant®.

### Process According to the Invention

The process according to the invention enables in particular the formation of secondary and/or tertiary alkylamines. The amine formed is preferably of general formula (A) below:

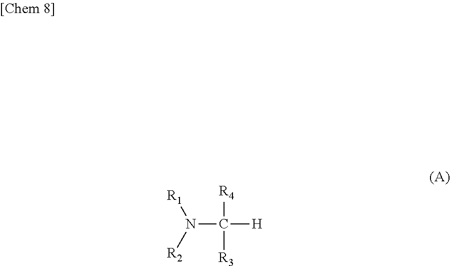

R₁ represents a linear, branched or cyclic alkyl radical comprising from 1 to 10 carbon atoms, preferably from 1 to 7 carbon atoms, more preferably from 1 to 4 carbon atoms, which is optionally substituted (preferably by an aryl radical such as a phenyl);
R₂ is selected from a hydrogen atom and a linear, branched or cyclic alkyl radical comprising from 1 to 10 carbon atoms, preferably from 1 to 7 carbon atoms, more preferably from 1 to 4 carbon atoms, which is optionally substituted (preferably by an aryl radical such as a phenyl); or else
R₁ and R₂, together and with the nitrogen atom carrying them, form a saturated or partially or totally unsaturated cyclic radical which is optionally substituted and may comprise one or more heteroatoms selected from oxygen and nitrogen; said cyclic moiety may comprise a number of ring members of between 3 and 9, preferably 5 or 6 ring members;
R₃ represents a linear, branched or cyclic, aromatic or non-aromatic hydrocarbon chain comprising from 1 to 10 carbon atoms, preferably from 1 to 7 carbon atoms, more preferably from 1 to 4 carbon atoms, which is optionally substituted (preferably by an aryl radical such as a phenyl);
R₄ is selected from a hydrogen atom and a linear, branched or cyclic, aromatic or non-aromatic hydrocarbon chain comprising from 1 to 10 carbon atoms, preferably from 1 to 7 carbon atoms, more preferably from 1 to 4 carbon atoms, which is optionally substituted (preferably by an aryl radical such as a phenyl);
or else
R₃ and R₄, together and with the carbon atom carrying them, form a saturated or partially unsaturated cyclic radical which is optionally substituted and may comprise one or more heteroatoms selected from oxygen and nitrogen; said cyclic moiety comprises a number of ring members of between 3 and 9, preferably 5 or 6 ring members.

R₁ and/or R₂ when represented by an alkyl radical as defined above may be substituted by one or more aryl groups containing between 6 and 10 carbon atoms, preferably a phenyl.

R₃ and/or R₄ when represented by an alkyl radical as defined above may be substituted by one or more aryl groups containing between 6 and 10 carbon atoms, preferably a phenyl.

R₃ and R₄ when, together and with the carbon atom which carries them, they form a saturated or partially unsaturated cyclic radical may be substituted by one or more alkyl groups comprising between 1 and 10 carbon atoms, preferably by one or more methyl groups.

In particular, R₁ represents a linear or branched alkyl radical comprising from 1 to 10 carbon atoms, preferably from 1 to 7 carbon atoms, more preferably from 1 to 4 carbon atoms;
R₂ is selected from a hydrogen atom and a linear or branched alkyl radical comprising from 1 to 10 carbon atoms, preferably from 1 to 7 carbon atoms, more preferably from 1 to 4 carbon atoms;
R₃ represents a linear or branched alkyl radical comprising from 1 to 10 carbon atoms, preferably from 1 to 7 carbon atoms, more preferably from 1 to 4 carbon atoms; and
R₄ is selected from a hydrogen atom and a linear or branched alkyl radical comprising from 1 to 10 carbon atoms, preferably from 1 to 7 carbon atoms, more preferably from 1 to 4 carbon atoms.

R₂ and/or R₄ is preferably a hydrogen atom.

More particularly, the amine formed is selected from the group consisting of:
diisopropylamine (DIPA), di-n-propylamine (DPA), N-ethylmethylamine (EMA), N-isopropylmethylamine, N-ethylpropylamine, N-ethylisopropylamine, N-ethylbutylamine, N-methylcyclohexylamine, N-ethylcyclohexylamine, N-ethylbenzylamine, N,N-dimethylethylamine (DMEA), N,N-dimethylisopropylamine (DMIPA), N,N-dimethylpropylamine (DMPA), N,N-dimethylbutylamine, N,N-diethylmethylamine (DEMA), triethylamine (TEA) and di-sec-butylamine (DB2A).

More particularly still, the amine formed is selected from the group consisting of DIPA, DMEA, DMIPA and EMA, more preferably DIPA and EMA.

Said aminating step may especially correspond to one or more of the following reactions:
the reaction of ammonia with a primary or secondary alcohol and/or a ketone to form primary, secondary and tertiary amines, preferably predominantly a secondary amine;
the reaction of a primary amine with a primary or secondary alcohol and/or a ketone to form secondary and tertiary amines, preferably predominantly a secondary amine; or
the reaction of a secondary amine with a primary or secondary alcohol and/or a ketone to form a tertiary amine.

In particular, in the context of the present invention, the desire is to form secondary and/or tertiary amines, preferably secondary amines.

Use is made in particular of an alcohol of formula (I) and/or a ketone of formula (II) with ammonia or an amine of formula (III):

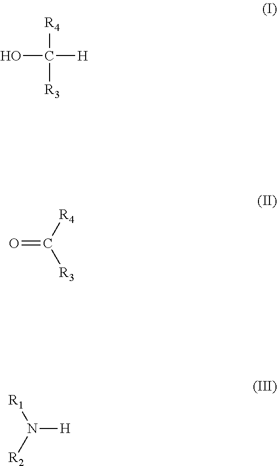

in which R₁, R₂, R₃ and R₄ are as defined above,
where R₄ is other than the hydrogen atom for the ketone of formula (II).

The alcohols of formula (I) include the following: ethanol, n-propanol, isopropanol, n-butanol, isobutanol, 2-butanol, n-pentanol, n-hexanol, methyl isobutyl carbinol, n-heptanol, 2-ethylhexanol, n-octanol, diisobutylcarbinol, cyclohexanol, benzyl alcohol, 2-phenylethanol and 3,3,5-trimethylcyclohexanol.

The ketones of formula (II) include the following: acetone, methyl ethyl ketone (MEK), methyl propyl ketone, methyl isopropyl ketone, diethyl ketone, methyl isobutyl ketone (MIBK), diisobutyl ketone, cyclobutanone, cyclopentanone, cyclohexanone, acetophenone, isophorone and 3,3,5-trimethylcyclohexanone.

It is understood that under the operating conditions of the aminating step, the alcohols undergo dehydration to form ketones, before reacting with hydrogen and the nitrogenous reactant. Apart from ammonia, the preferred aminic reactants of formula (III) are the following: methylamine, dimethylamine, ethylamine, diethylamine, n-propylamine, di-n-propylamine, isopropylamine, diisopropylamine, n-butylamine, di-n-butylamine, isobutylamine, 2-butylamine, pyrrolidine, piperidine, morpholine, cyclohexylamine, benzylamine and 2-phenylethylamine.

Examples of secondary or tertiary amines that may thus be prepared preferably according to the process of the invention are:
diisopropylamine (DIPA) starting from acetone and/or isopropanol and ammonia,
di-n-propylamine (DPA) starting from propanol and ammonia,
di-sec-butylamine starting from methyl ethyl ketone and/or 2-butanol with ammonia,
N-ethylmethylamine (EMA) starting from ethanol and monomethylamine (MMA),
N-isopropylmethylamine starting from acetone and/or isopropanol and MMA,
N-ethylpropylamine starting from n-propanol and monoethylamine or else starting from ethanol and n-propylamine,
N-ethylisopropylamine starting from ethanol and isopropylamine or else starting from acetone and/or isopropanol and monoethylamine,
N-ethylbutylamine starting from n-butanol and monoethylamine or else starting from ethanol and n-butylamine,
N-methylcyclohexylamine starting from cyclohexanol and/or cyclohexanone and MMA,
N-ethylcyclohexylamine starting from cyclohexanol and/or cyclohexanone and monoethylamine or else starting from ethanol and cyclohexylamine,
N-ethylbenzylamine starting from benzyl alcohol and monoethylamine or else starting from ethanol and benzylamine,
N,N-dimethylethylamine (DMEA) starting from ethanol and dimethylamine (DMA),
N, N-dimethylisopropylamine (DMIPA) starting from acetone and/or isopropanol and DMA,
N, N-dimethylpropylamine (DMPA) starting from n-propanol and DMA,
N, N-dimethylbutylamine starting from n-butanol and DMA,
N, N-diethylmethylamine (DEMA) starting from ethanol and DMA.

The aminating step according to the invention enables the formation of secondary and/or tertiary amines (and water) in the gaseous phase and in the presence of hydrogen. Said process may be carried out batchwise or continuously, preferably continuously.

“Gas phase” or “gaseous phase” means in particular that the reactants (alcohol and/or ketone and nitrogenous reactant) are in the gaseous state, under the temperature and pressure conditions of said aminating step. The reactor may be supplied with gaseous phase by passage of the liquid reactants through an evaporator (heated for example by steam or by any other known means) beforehand. The evaporator temperature is set so as to ensure that the reactants pass from the liquid to the gaseous state under the pressure conditions employed. The gases formed may then be carried towards the entry of the reactor, for example with the stream of hydrogen and, where appropriate, ammonia.

The catalytic reaction may be operated under hydrogen pressure (H₂), preferably in excess. The molar ratio of hydrogen to alcohol and/or ketone is preferentially between 0.5 and 20 mol/mol, preferably between 1 and 15 mol/mol and more preferentially between 2 and 10 mol/mol.

The amination reaction is preferably performed through one or more fixed beds of catalyst like that according to the invention. The fixed bed or beds may comprise one or more layers of catalyst according to the invention. Where a catalytic bed is used that comprises multiple layers of catalysts, the concentration of metal (for example, of Cu and/or of Mn) may increase from the entry to the exit of the reactor, and the number of layers may vary as a function of the length of the catalytic bed.

The amination reaction may be carried out within a (or two or more) tubular or multi-tubular reactor(s), in series or in parallel.

The amination reaction may be carried out under an absolute pressure in the reactor of less than or equal to 30 bar, preferably between 1 and 20 bar and more preferably between 2 and 10 bar.

The amination reaction may be carried out at a temperature of between 120° C. and 220° C., preferably between 140° C. and 200° C. and more preferably between 150° C. and 190° C.

The temperature of the reactor may be maintained by means of a heat transfer fluid, which may be heated by steam, electrically or by any other known means and may be cooled by means of a refrigeration circuit with water and/or ethylene glycol or any other known refrigeration fluid. The heat transfer fluid may in particular comprise a mixture of molten nitrate salts (KNO₃, NaNO₃, LiNO₃).

The amination reaction may be performed with a molar ratio of alcohol and/or ketone to nitrogenous reactant of between 0.1 and 20 mol/mol, preferably between 0.5 and 10 mol/mol and more preferentially between 1 and 5 mol/mol.

The mass flow rate of alcohol and/or of ketone per unit volume of catalytic bed (MVH) may be between 0.05 and 1.0 kg/L·h, preferably between 0.10 and 0.80 kg/L·h and more preferentially between 0.15 and 0.60 kg/L·h.

The preparation process according to the invention may also comprise the following steps:
i) an aminating step as defined above, said step producing a departing stream G comprising a secondary and/or tertiary amine and water;
ii) at least one step of separating the stream G, to give:
   a stream comprising water, and
   a stream comprising the secondary amine and/or the tertiary amine;
iii) optionally, a step of separating the stream comprising both a secondary amine and a tertiary amine, to give:
   a stream comprising the secondary amine; and
   a stream comprising the tertiary amine; and
iv) optionally, recycling the stream comprising the tertiary amine to step i).

The secondary and/or tertiary amines may then be recovered and optionally purified.

More particularly, said process may comprise the following steps:
a) an aminating step as defined above, said step producing a departing stream G in the gaseous state comprising a secondary and/or tertiary amine, water and unreacted hydrogen;
b) condensing and separating said stream G, to give:
   a liquid stream G′ comprising a secondary and/or tertiary amine and water, and
   a gaseous hydrogen stream G″;
c) optionally, recycling the stream G″ to step a);
d) separating the stream G′, to give:
   a stream K comprising water, and
   a stream L comprising the secondary amine and/or the tertiary amine;
e) optionally, separating the stream L when it comprises both a secondary amine and a tertiary amine, to give:
   a stream M comprising the secondary amine; and
   a stream N comprising the tertiary amine; and
f) optionally, recycling the stream N to step a).

Steps b) and d) may or may not be simultaneous.

The gaseous stream G″ may comprise traces of secondary and/or tertiary amines and possibly of primary amines.

When ammonia is used as a reactant and does not react completely, it may be found in stream G″ and also, in trace form, in G′. In that case, it is possible to conduct an auxiliary separation of stream G′ and/or G″ to recover the ammonia and recycle it to step a).

When ammonia is used as a reactant, moreover, the corresponding primary amine may also be formed as a by-product. This primary amine is found successively in streams G, G′ and L (and possibly in G″ in trace form). It may be separated from the stream L at the end of the separating step e) to give a stream P comprising it. Said stream P may be recycled to the aminating step a).

Separating steps b), d) and e) may take place by any known means (for example by distillation or settling) and preferably by distillation.

The secondary and/or tertiary amines produced may be subsequently purified if necessary.

Use is made in particular of a fractional distillation by means of a series of distillation columns operating continuously.

More particularly, said process may comprise the following steps:
a) an aminating step as defined above, said step producing a departing stream G in the gaseous state comprising a secondary and/or tertiary amine, water and possibly the unreacted reactants, and the alcohol resulting from the hydrogenation reaction of the ketone to alcohol;
b) condensing and separating said stream G, to give:
   a liquid stream G′ comprising a secondary and/or tertiary amine, water and possibly the unreacted reactants, and the alcohol resulting from the hydrogenation reaction of the ketone to alcohol, and
   a gaseous hydrogen stream G″ comprising traces of secondary and/or tertiary amine, and possibly traces of unreacted reactants, and also traces of the alcohol resulting from the hydrogenation reaction of the ketone to alcohol;
c) optionally, recycling the stream G″ to step a);
d) separating the stream G′, to give:
   a stream K comprising water and possibly the unreacted reactants, and the alcohol resulting from the hydrogenation reaction of the ketone to alcohol, and
   a stream L comprising the secondary amine and/or the tertiary amine;
e) optionally, separating the stream L when it comprises both a secondary amine and a tertiary amine, to give:
   a stream M comprising the secondary amine; and
   a stream N comprising the tertiary amine; and
f) optionally, recycling the stream N to step a).

The stream K may also be separated to give a stream O comprising water and a stream T comprising the alcohol. The alcohol thus recovered may be recycled to step a).

### Optional Activation of the Catalyst

The catalyst may be activated prior to step a). The reason is that the catalysts are generally charged to the reactor in oxidized or pre-reduced form (meaning that the metals, such as Cu and Mn, are wholly or partly in the form of oxides). In this case, the catalyst is preferably activated beforehand. The activation takes place by reduction, preferably in the reactor in which the aminating step is to be carried out (activation in situ). The catalyst is activated by conventional methods, well known to those skilled in the art. It affords the metallic species which are active for hydrogenation or dehydrogenation by reduction of the corresponding oxidized forms. The copper thus passes from the Cu^{∥} state (in CuO) to the Cu⁰ state via the following reaction: CuO+H₂→Cu+H₂O

The catalyst may therefore be activated in a stream of hydrogen (H₂) at a temperature of between 150° C. and 400° C., for example between 200° C. and 400° C., preferably between 250° C. and 350° C.

### Use According to the Invention

The present invention also relates to the use of a catalyst as defined above for a process for preparing secondary and/or tertiary amines as defined above, and particularly for the aminating step as described above.

### EXAMPLES

### Abbreviations and Definitions:

ACE: acetone
ISO: isopropanol
EtOH: ethanol
MIPA: monoisopropylamine
DIPA: diisopropylamine
EMA: N-ethylmethylamine
DEMA: N,N-diethylmethylamine
DMEA: N,N-dimethylethylamine
DMIPA: N,N-dimethylisopropylamine
RM: molar ratio
MVH: mass hourly flow rate of the feed per unit volume of catalyst (unit: kg/L·h)
S_{A}=selectivity for amine (A) produced relative to reactants converted

The selectivities are calculated on the basis of the mass compositions of the crude mixtures exiting the reaction zone, the compositions being determined by gas-chromatographic analyses.

$${DC}_{DMA} = degree of conversion of DMA employed = conversion of the DMA$$

NL: normal litre, corresponding to a volume of 1 L under standard conditions of pressure (1.013 bar) and temperature (273 K).

### Example 1: Synthesis of Diisopropylamine (DIPA)—Secondary Amine

The tests are carried out in a vertical tubular reactor containing a catalytic bed with a volume of 7 L and a length of 2.8 m. The reactor is immersed in a bath of molten nitrate salts (KNO₃, NaNO₃, LiNO₃) which is heated electrically and cooled by means of circulation of water through a cooling pin. A temperature probe inserted and able to slide within a sheath traversing the whole of the catalytic bed allows the reaction temperature to be measured.

### Nickel Catalyst (Comparative):

The three-layer catalytic bed comprises a catalyst based on nickel in the form of cylindrical pellets (4.8×4.8 mm) whose composition by weight before activation is as follows:
bottom layer (reactor entry)≈0.33 L:
5.3% Ni (in Ni and NiO form) on Al₂O₃ and 2.5-5% of graphite,
middle layer=0.33 L:
20% Ni (in Ni and NiO form) on Al₂O₃ and 2.5-5% of graphite,
top layer (reactor exit)≈0.33 L: 43% Ni (in Ni and NiO form) on Al₂O₃ and 10% of graphite.

### Copper Catalyst C1 (of the Invention):

The single-layer catalytic bed comprises cylindrical pellets (4.8×4.8 mm) of a manganese-doped copper-based catalyst on an alumina support (Al₂O₃), the copper and the manganese being in oxidized form before activation.

The copper concentration of the catalyst by weight is 46% (corresponding to 57.6% expressed as CuO) and the manganese concentration by weight is 6% (corresponding to 9.5% expressed as MnO₂) before activation.

### Activation of the Nickel Catalyst and of Catalyst C1:

The tubular reactor, preheated to 240° C. and at atmospheric pressure, is charged with a stream of hydrogen and nitrogen with volume flow rates per unit volume of catalytic bed (HSV) respectively of 50 NL/L·h of H₂ and 500 NL/L·h of N₂. As soon as the zone of maximum heat production, monitored by the multi-point temperature probe, has passed through the whole of the catalytic bed (after around 8 h), the introduction of nitrogen is halted and the injection of hydrogen is continued for 12 h with an increase in the temperature of the reactor to 280° C. in the case of the copper catalyst and to 350° C. in the case of the nickel catalyst and with an HSV of H₂ of 100 NL/L·h.

### Aminating Step:

The reactor is then fed from bottom to top with a mixture of fresh acetone, recycled isopropanol, ammonia and hydrogen which has been previously evaporated and preheated through a steam exchanger. The pressure of the reactor is kept at 4 bar absolute and the temperature at 150° C. The table below indicates the results obtained according to the nature of the catalytic bed, the proportion of recycled isopropanol, the MVH and the RM of NH₃ and H₂:

TABLE 1

| | Operating | Molar flow rate (mol/h) | | MVH | RM | RM | | | | DIPA |
|---|---|---|---|---|---|---|---|---|---|---|
| | time | ACE | ISO | [ACE + ISO] | (ACE + ISO)/ | H2/ACE + | Selectivities (%) | | | productivity |
| Catalyst | (h) | fresh | ‘recycled’ | (kg/ [symbol] h) | NH3 | ISO | MIPA | DIPA | [symbol] | (kg/ [symbol] h) |
| Cata Ni | [symbol] | 21 | 21 | 0.35 | 0.5 | 4 | 61.07 | 38.26 | 0.21 | [symbol] |
| (not of the invention) | 1976 | 29 | 13 | [symbol] | 0.5 | 4 | 52.07 | 46.51 | 0.12 | 0.116 |
| Cata Cu (C1) | 273 | 21 | 21 | 0.35 | 0.5 | 4 | [symbol] | [symbol] | — | [symbol] |
| doped with Mn | [symbol] | 38 | 4 | [symbol] | 1 | 5 | 26.04 | 72.71 | — | 0.180 |
| | [symbol] | 38 | 4 | 0.35 | 2 | [symbol] | [symbol] | 86.75 | — | [symbol] |
| | 1898 | 14 | 7 | 0.18 | [symbol] | 11 | 22.35 | 77.58 | — | 0.094 |
| | 1921 | 14 | 7 | 0.18 | 2 | 5.5 | [symbol] | 89.25 | — | 0.083 |
| | 1946 | 14 | 7 | 0.18 | 2 | 11.5 | 10.05 | 89.74 | — | 0.083 |

[symbol]

indicates data missing or illegible when filed

Catalyst C1 affords a much greater selectivity for diisopropylamine than does the nickel catalyst, and does so without secondary formation of EIPA, which is difficult to separate from the DIPA by distillation. A selectivity for DIPA of close to 90% can be obtained directly without recycling of MIPA.

### Example 2: Synthesis of Dimethylisopropylamine (DMIPA)—Tertiary Amine

These tests were carried out in a thermally regulated vertical tubular reactor containing a catalytic bed comprising catalyst C1 or a catalyst C2 with a volume of 1 L and a length of 80 cm.

Copper catalyst C1 (of the invention): as described in Example 1
Copper catalyst C2 (comparative):
The catalytic bed is made of cylindrical pellets (6×5 mm) whose composition by weight before activation is as follows: 76% CuO, 3% MgO, 1.5% Cr₂O₃, on silica (SiO₂).

### Aminating Step:

After prior activation of the catalysts by reduction with H₂ at 250-350° C., the reactor is fed from bottom to top with a mixture of fresh acetone and/or fresh and/or recycled isopropanol, DMA and hydrogen which has been previously evaporated and preheated through an electrically heated exchanger. The synthesis is operated with a large molar excess of acetone and/or isopropanol relative to the DMA, under a pressure of 8 bar and at a temperature of 185° C. The table below indicates the results obtained according to the nature of the catalytic bed and the respective molar flow rates of ACE+ISO and DMA, given the fact that in each case the conversion of the DMA is greater than 99%:

TABLE 2

| | Operating | | | MVH | MVH | RM | RM | | | | |
|---|---|---|---|---|---|---|---|---|---|---|---|
| | time | Molor flow rate (mol/h) | | [ACE + ISO] | [DMA] | (ACE + ISO)/ | H2/(ACE + | Selectivities [symbol] (%) | | | |
| Catalyst | (h) | ACE | ISO | (kg/ [symbol] h) | (kg/ [symbol] h) | DMA | ISO) | TMA | Me-IPA | DMIPA | [symbol] -DIPA |
| Cata Cu (C2) | [symbol] | 2.7 | 6.6 | 0.553 | 0.117 | [symbol] | 7.0 | 4.52 | [symbol] | 85.99 | 3.08 |
| (not of the | 257 | [symbol] | 4.64 | 0.387 | 0.083 | 3.5 | 8.0 | 4.45 | [symbol] | 86.00 | 2.90 |
| invention) | 234 | 2.7 | 6.6 | [symbol] | 0.120 | 3.5 | 7.0 | 1.38 | [symbol] | [symbol] | 0.80 |
| Cata Cu (C1) | 298 | [symbol] | 4.64 | 0.387 | 0.083 | 3.5 | 8.0 | 1.31 | 1.71 | [symbol] | [symbol] |
| doped with Mn | [symbol] | 1.96 | 4.64 | 0.387 | 0.083 | 3.5 | 8.0 | 1.38 | 1.82 | 94.61 | 0.92 |

[symbol]

indicates data missing or illegible when filed

With catalyst C2, the selectivity for DMIPA relative to DMA is 8 to 9% less than that obtained with catalyst C1. This may be because of a greater dismutation of the DMA into TMA and MMA; the MMA subsequently reacts with the acetone to form methylisopropylamine (Me-IPA) and methyldiisopropylamine (Me-DIPA).

### Example 3: Synthesis of Ethylmethylamine (EMA—Secondary Amine) and/or Diethylmethylamine (DEMA—Tertiary Amine) from Ethanol and MMA, with or without Recycling of DEMA

These tests were carried out in the same apparatus as that of Example 2 with the previously reduced catalyst C1.

The synthesis is operated with a molar excess of ethanol relative to the MMA, in the presence of hydrogen, under a pressure of 8 bar and at a temperature of 175° C., and where applicable with recycling of DEMA.

The table below indicates the results obtained according to the EtOH/MMA molar ratio and the optional recycling of DEMA:

TABLE 3

| | Operating | Molar flow rate (mol/h) | | | MVH | MVH | | | | | EMA |
|---|---|---|---|---|---|---|---|---|---|---|---|
| | time | | | DEMA | [EtOH] | [MMA] | RM | RM | Selectivities [symbol] (%) | | productivity |
| Catalyst | (h) | EtOH | MMA | recycled | (kg/ [symbol] h) | (kg/ [symbol] h) | EtOH/MMA | H2/EtOH | EMA | DEMA | (kg/ [symbol] h) |
| Cata Cu (C1) | 139 | 4.5 | 3.5 | — | 0.207 | 0.109 | 1.3 | 8.4 | 75.45 | 19.52 | 0.120 |
| doped with Mn | 480 | 4.5 | 3.0 | — | 0.207 | 0.093 | 1.5 | 14.2 | 79.12 | [symbol] | 0.108 |
| | 512 | 4.5 | 2.9 | 1.09 | 0.207 | 0.089 | 1.6 | 14.2 | 85.35 | 10.41 | 0.107 |
| | 760 | 4.5 | 3.0 | 1.65 | 0.207 | 0.093 | 1.5 | 14.2 | 92.73 | 3.57 | 0.097 |

[symbol]

indicates data missing or illegible when filed

The results at 512 h and 760 h of operation correspond to the tests carried out with recycling of the DEMA recovered at the end of the reaction by distillation and reintroduced into the reactor.

It is apparent that, depending on the flow rate of tertiary amine (DEMA) recycled, the selectivity for secondary amine (EMA) relative to the MMA may become greater than 90%.

### Example 4: Synthesis of the Secondary Amine Ethylpropylamine (EPA) from Ethanol and MEA

These tests were carried out in the same apparatus as that of Example 2 with the previously reduced catalyst C1.

A continuous feed is supplied of 6 mol/h of ethanol and 2 mol/h of monoethylamine (MEA), at a temperature of 170° C., in the presence of H₂ (RM H₂/EtOH=4), under a pressure of 4 bar. The conversion of MEA at the reactor exit is 81% and the EPA is obtained with a selectivity of 92% relative to the MEA converted.

### Example 5: Synthesis of the Secondary Amine di-n-propylamine (DPA) from n-propanol and Ammonia, with Recycling of n-PA (n-propylamine) and TPA (rripropylamine)

These tests were carried out in the same apparatus as that of Example 2 with the previously reduced catalyst C1.

With a continuous feed of 8 mol/h of n-propanol (MVH=0.48 kg/L·h) and 24 mol/h of ammonia (RM PrOH/NH₃=0.33), at a temperature of 165° C., in the presence of H₂ (RM H₂/PrOH=4), under a pressure of 4 bar, and with recycling of 196 g/h of n-PA and 90 g/h of TPA, the DPA is obtained with a selectivity of 92.5% for a conversion of n-propanol of 79.0%.

### Example 6: Synthesis of the Tertiary Amine Dimethylpropylamine (DMPA) from n-Propanol and DMA

These tests were carried out in the same apparatus as that of Example 2 with the previously reduced catalyst C1, under an absolute pressure of 8 bar and at a reaction temperature TR of 185 or 190° C.

The table below indicates the results obtained according to the nature of the catalytic bed and the reaction conditions employed:

TABLE 4

| | | | MVH | | | | | | | |
|---|---|---|---|---|---|---|---|---|---|---|
| | PrOH flow rate | MVH [PrOH] | [DMA] | RM | RM | Tg | DC [symbol] | Selectivities [symbol] (%) | | |
| Catalyst | (mol/h) | (kg/ [symbol] h) | (kg/ [symbol] h) | PrOH/DMA | H2/PrOH | (° C.) | (%) | TMA | DMPA | Me-DPA |
| Cata Cu (C1) | 4.1 | 0.25 | 0.088 | 2.1 | 4.7 | 190 | 99.46 | 0.65 | 98.37 | 0.56 |
| doped with Mn | 6.0 | 0.36 | 0.108 | 2.5 | 5.1 | 185 | 99.39 | 0.40 | 98.24 | 0.54 |
| | 6.0 | 0.36 | 0.104 | 2.6 | 5.1 | 190 | 99.73 | 0.34 | 98.61 | 0.42 |
| | 6.0 | 0.36 | 0.082 | 3.3 | 5.4 | 185 | 99.89 | 0.27 | 98.68 | 0.35 |

[symbol]

indicates data missing or illegible when filed

With catalyst C1, very high selectivities for DMPA are obtained, of more than 98%, with a degree of conversion of the DMA of more than 99% (DC), with very low levels of aminic impurities.

### Example 7: Synthesis of the Tertiary Amine Dimethylethylamine (DMEA) from Ethanol and DMA-Alternation of Syntheses

These tests were carried out in the same apparatus as that of Example 1 but with a catalytic bed of catalyst C1 with a volume of 3.2 L and a length of 2.8 m and under the following reaction conditions:
Mean molar flow rate of REN grade ethanol containing 4.3% water: 28.8 mol/h, corresponding to a mean MVH of ethanol of 0.435 kg/L_{cata.}h
Mean molar flow rate of DMA: 6.6 mol/h, corresponding to a mean MVH of DMA of 0.135 kg/L_{cata.}h and to an EtOH/DMA mean molar ratio of 3
Mean molar ratio H₂/EtOH=8
Reaction performed under an absolute pressure of 8 bar

The catalytic bed is operated alternately for production of DMEA and for production of DMIPA in order to evaluate the stability of the catalyst after different production campaigns.

The table below indicates the corresponding selectivities relative to DMA; the selectivities for just the DMEA production campaigns are calculated below.

TABLE 5

| | Operating | | | | | |
|---|---|---|---|---|---|---|
| | time | Tg | DC_{DMA} | Selectivities_{/DMA} (%) | | |
| | (h) | (° C.) | (%) | TMA | DMEA | DEMA |
| Campaign 1 | 78 | 190 | 99.95 | 0.64 | 98.21 | 0.83 |
| | 260 | 180 | 99.97 | 0.75 | 98.00 | 0.84 |
| | 332 | 190 | 99.97 | 0.62 | 98.12 | 0.86 |
| | Use of catalytic bed for synthesis | | | | | |
| | of DMIPA from acetone and DMA for 888 h | | | | | |
| Campaign 2 | 1260 | 190 | 99.96 | 0.78 | 97.67 | 1.00 |
| | 1314 | 185 | 99.97 | 0.64 | 98.08 | 0.81 |
| | 1361 | 175 | 99.96 | 0.69 | 97.97 | 0.76 |
| | 1403 | 165 | 97.79 | 0.87 | 97.60 | 0.78 |
| | 1490* | 190 | 99.94 | 0.94 | 97.63 | 1.04 |
| | Use of catalytic bed for synthesis of | | | | | |
| | DMIPA from acetone and DMA for 650 h | | | | | |
| | Then: Regeneration of the catalytic | | | | | |
| | bed via an operation of oxidation | | | | | |
| | with air followed by a step of reduction with hydrogen | | | | | |
| Campaign 3 | 2185 | 180 | 99.97 | 0.84 | 97.73 | 1.07 |
| | 2259 | 180 | 99.95 | 0.83 | 97.89 | 0.94 |

*using recycled ethanol containing 10.7% water

These results demonstrate the stability of catalyst performance over time, in spite of the intermediate operating campaigns for DMIPA starting from acetone (a much more exothermic reaction). This catalyst can therefore be employed advantageously in a multi-purpose production unit in which amines of different kinds can be manufactured by successive campaigns.

It is also apparent that, if needed, the catalyst can be easily regenerated by a step of oxidation followed by renewed reduction with hydrogen, without loss of performance.

1. Process for preparing secondary and/or tertiary amines, comprising an aminating step, said aminating step being carried out by gas-phase reaction of a primary or secondary alcohol and/or of a ketone with ammonia or a primary or secondary amine, in the presence of a catalyst and hydrogen,
said catalyst comprising copper doped with manganese, and
the amount of manganese being between 1% and 10% by weight, relative to the total weight of the catalyst.

2. Preparation process according to claim 1, in which the amount of copper in the catalyst is between 20% and 60% by weight, more preferably between 35% and 50% by weight, relative to the total weight of the catalyst.

3. Preparation process according to claim 1, in which the amount of manganese is between 4% and 10% by weight, preferably between 4% and 8% by weight, relative to the total weight of the catalyst.

4. Preparation process according to claim 1, in which the catalyst comprises a support selected from the group consisting of alumina (Al₂O₃), silica (SiO₂), titanium dioxide, zirconia, and mixtures of two or more thereof, preferably alumina and/or silica.

5. Preparation process according to claim 1, in which the amine formed is of general formula (A) below: 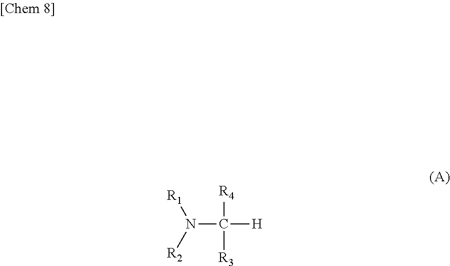
in which:
R₁ represents a linear, branched or cyclic alkyl radical comprising from 1 to 10 carbon atoms, preferably from 1 to 7 carbon atoms, more preferably from 1 to 4 carbon atoms, which is optionally substituted;
R₂ is selected from a hydrogen atom and a linear, branched or cyclic alkyl radical comprising from 1 to 10 carbon atoms, preferably from 1 to 7 carbon atoms, more preferably from 1 to 4 carbon atoms, which is optionally substituted;
or else
R₁ and R₂, together and with the nitrogen atom carrying them, form a saturated or partially or totally unsaturated cyclic radical which is optionally substituted and may comprise one or more heteroatoms selected from oxygen and nitrogen; the cyclic moiety may comprise a number of ring members of between 3 and 9, preferably 5 or 6 ring members;
R₃ represents a linear, branched or cyclic, aromatic or non-aromatic hydrocarbon chain comprising from 1 to 10 carbon atoms, preferably from 1 to 7 carbon atoms, more preferably from 1 to 4 carbon atoms, which is optionally substituted;
R₄ is selected from a hydrogen atom and a linear, branched or cyclic, aromatic or non-aromatic hydrocarbon chain comprising from 1 to 10 carbon atoms, preferably from 1 to 7 carbon atoms, more preferably from 1 to 4 carbon atoms, which is optionally substituted;
or else
R₃ and R₄, together and with the carbon atom carrying them, form a saturated or partially unsaturated cyclic radical which is optionally substituted and may comprise one or more heteroatoms selected from oxygen and nitrogen; said cyclic moiety comprises a number of ring members of between 3 and 9, preferably 5 or 6 ring members.

6. Preparation process according to claim 1, in which the amine formed is selected from the group consisting of:
diisopropylamine (DIPA), di-n-propylamine (DPA), N-ethylmethylamine (EMA), N-isopropylmethylamine, N-ethylpropylamine, N-ethylisopropylamine, N-ethylbutylamine, N-methylcyclohexylamine, N-ethylcyclohexylamine, N-ethylbenzylamine, N,N-dimethylethylamine (DMEA), N, N-dimethylisopropylamine (DMIPA), N,N-dimethylpropylamine (DMPA), N, N-dimethylbutylamine, N, N-diethylmethylamine (DEMA), triethylamine (TEA) and di-sec-butylamine (DB2A).

7. Preparation process according to claim 1, in which the amine formed is selected from the group consisting of DIPA, DMEA, DMIPA and EMA, more preferably DIPA and EMA.

8. Preparation process according to claim 1, in which the catalyst is activated beforehand by reduction, preferably in a stream of hydrogen (H₂), at a temperature of between 150° C. and 400° C.

9. Preparation process according to claim 1, comprising the following steps:
i) an aminating step as defined, said step producing a departing stream G comprising a secondary and/or tertiary amine and water;
ii) at least one step of separating the stream G, to give:
a stream comprising water, and
a stream comprising the secondary amine and/or the tertiary amine;
iii) optionally, a step of separating the stream comprising both a secondary amine and a tertiary amine, to give:
a stream comprising the secondary amine; and
a stream comprising the tertiary amine; and
iv) optionally, recycling the stream comprising the tertiary amine to step i).

10. Preparation process according to claim 9, comprising the following steps:
a) an aminating step as defined, said step producing a departing stream G in the gaseous state comprising a secondary and/or tertiary amine, water and unreacted hydrogen;
b) condensing and separating said stream G, to give:
a liquid stream G′ comprising a secondary and/or tertiary amine and water, and
a gaseous hydrogen stream G″;
c) optionally, recycling the stream G″ to step a);
d) separating the stream G′, to give:
a stream K comprising water, and
a stream L comprising the secondary amine and/or the tertiary amine;
e) optionally, separating the stream L when it comprises both a secondary amine and a tertiary amine, to give:
a stream M comprising the secondary amine; and
a stream N comprising the tertiary amine; and
f) optionally, recycling the stream N to step a).

11. Process according to claim 10, in which, when the reactant is ammonia, the stream G also comprises a primary amine as by-product, said primary amine being present subsequently in streams G′ and L and being separated in a stream P at the end of the separating step e), where said stream P may optionally be recycled to step a).

12. A catalyst comprising copper doped with manganese, the manganese being present in an amount of between 1% and 10% by weight, relative to the total weight of the catalyst, for preparing secondary and/or tertiary amines.
